(19) United States
(12) Patent Application Publication
(10) Pub. No.: US 2025/0009960 A1
(43) Pub. Date: Jan. 9, 2025

(54) MANUFACTURING METHOD FOR BACKFLOW TYPE IRRIGATION NEEDLE TIP, AND BACKFLOW TYPE IRRIGATION NEEDLE TIP

(71) Applicant: Xu He, Shanghai Province (CN)

(72) Inventors: Xu He, Shanghai Province (CN); Shiliang Yuann, Shanghai Province (CN); Xiaolei Zhu, Shanghai Province (CN); Li Wang, Shanghai Province (CN); Yutao Wang, Shanghai Province (CN); Hao Wang, Shanghai Province (CN); Gongxu Luo, Shanghai Province (CN); Jia He, Shanghai Province (CN)

(21) Appl. No.: 18/892,723

(22) Filed: Sep. 23, 2024

Related U.S. Application Data
(63) Continuation of application No. PCT/CN2023/082681, filed on Mar. 21, 2023.

(30) Foreign Application Priority Data
Mar. 25, 2022 (CN) 202220669713.7
Apr. 8, 2022 (CN) 202210380250.7

(51) Int. Cl. A61M 3/02; A61C 17/02; B23K 26/384

(52) U.S. Cl. CPC A61M 3/0279; A61C 17/02; B23K 26/384; A61M 2207/00

(57) ABSTRACT

The disclosure provides a manufacturing method for a backflow type irrigation needle tip, and a backflow type irrigation needle tip. In the manufacturing method, a pointed portion of the irrigation needle tip is a closed end. The method includes: setting a laser parameter of the laser apparatus, where the laser parameter includes a repetition frequency, single pulse energy, and a focal spot size; setting a diameter parameter of a laser machining trajectory; adjusting a laser beam for aiming to a designated portion of the irrigation needle tip to be machined, and adjusting a direction of the laser beam and an axial direction of the irrigation needle tip to form an angle, where a range of the angle α is 10°≤α<90°; and activating the laser apparatus, and forming an inclined hole towards the closed end on a pipe wall of the irrigation needle tip.

### CROSS-REFERENCE TO RELATED PATENT APPLICATION

This application is a continuation application of International Application No. PCT/CN2023/082681, filed on Mar. 21, 2023, which itself claims priority to Chinese Patent Application No. 202220669713.7, filed on Mar. 25, 2022, and Chinese Patent Application No. 202210380250.7, filed on Apr. 8, 2022. The disclosures of the above-mentioned applications are hereby incorporated by reference.

### FIELD

The disclosure relates to the technical field of medical instruments, and in particular to a manufacturing method for a backflow type irrigation needle tip, and a backflow type irrigation needle tip.

### BACKGROUND OF THE INVENTION

Irrigation and disinfection, common steps in stomatological therapy, have been applied to in a wide range of fields such as surgical debridement, periodontal cleaning, and root canal therapy. In therapy, an injured wound and a root canal system communicate with internal structures, such as a bone marrow cavity, a maxillary sinus, and a neural canal. Owing to imbalance between an internal pressure and an external pressure, an irrigant and a root canal sealer at a relatively positive pressure may tend to enter a human body accidentally. With a fine and complex tissue structure and an internally-infected stubborn bacterial biofilm, the root canal system has high requirements on an irrigator for transporting a chemical solution. It is common practice to irrigate and disinfect the root canal system with a syringe and a needle tip, and lead in the root canal sealer with an injection system at a positive pressure. The needle tip includes an open-end needle tip and a closed-end needle tip, and has one or more outlets. However, the existing needle tip has an uncontrolled irrigant lead-out direction, a low speed, and undesirable safety when used for irrigation and disinfection. To control a solution ejection direction of an irrigation needle tip, a flow guide barrier is required to be designed and machined on the needle tip, so as to form a backflow type irrigation needle tip. Accordingly, a solution lead-out direction is controlled, and an effect of solution backflow and ejection is realized. In addition, the irrigation needle tip is difficult to machine because of its rigid material and small size.

### SUMMARY OF THE INVENTION

To solve the defects in the prior art, the disclosure provides a manufacturing method for a backflow type irrigation needle tip, and a backflow type irrigation needle tip.

One aspect of the present invention provides a manufacturing method for a backflow type irrigation needle tip, wherein a hole is punched on the irrigation needle tip through a laser apparatus, and a pointed portion of the irrigation needle tip is a closed end; and the method comprises:
step a: setting a laser parameter of the laser apparatus, wherein the laser parameter comprises a repetition frequency, single pulse energy, and a focal spot size;
step b: setting a diameter parameter of a laser machining trajectory;
step c: adjusting a laser beam for aiming to a designated portion of the irrigation needle tip to be machined, and adjusting a direction of the laser beam and an axial direction of the irrigation needle tip to form an angle, wherein a range of the angle α is 10°≤α<90°; and
step d: activating the laser apparatus, and forming an inclined hole towards the closed end on a pipe wall of the irrigation needle tip.

Preferably, in step a, the laser apparatus comprises a laser, an optical path reflection mirror, and a machining head.

Preferably, the laser and the machining head are controlled through a computer.

Preferably, in step b, the laser machining trajectory is circular and has a diameter of 90 microns-400 microns.

Preferably, in step c, a distance from the designated portion to the pointed portion of the irrigation needle tip is 0.07 millimeter-16 millimeters.

Preferably, the range of the angle α is 40°≤α≤70°.

Preferably, in step c, the irrigation needle tip is fixed to an auxiliary workpiece of a workbench, and a desired angle α is acquired by adjusting an angle between a surface of the auxiliary workpiece and a horizontal plane.

Another aspect of the present invention provides a backflow type irrigation needle tip, manufactured through the aforementioned manufacturing method, wherein a pointed portion of the irrigation needle tip is a closed end, an inclined hole towards the closed end is formed on a pipe wall of the irrigation needle tip, and a range of an included angle α between a hole forming direction of the inclined hole, i.e. a direction of a laser beam and an axial direction of the needle tip is 10°≤α<90°.

Preferably, the included angle α between the hole forming direction of the inclined hole and the axial direction of the needle tip is 45°.

Preferably, a distance from the inclined hole to the pointed portion of the irrigation needle tip is 0.07 millimeter-16 millimeters, and 1-6 inclined holes are provided.

The disclosure has the beneficial effects as follows: in the method of the disclosure, a hole is punched on a side wall of a needle pipe of the irrigation needle tip by a laser beam controlled through a computer system, so that a position, a size, and a direction of an inclined hole can be accurately designed and controlled. In other words, in the method of the disclosure, water outlet holes having different numbers, sizes, and directions may be punched on the side wall of the needle pipe of the irrigation needle tip according to clinical demands. Water outlet holes may also be individually manufactured according to different root canal shapes. In addition, by changing an opening angle of the inclined hole of the needle tip and a flow direction of an irrigant, the backflow type irrigation needle tip manufactured through the method of the disclosure may generate a large irrigation pressure and a high flow speed. Therefore, requirements of a large amount of rapid and safe irrigation can be satisfied, and when the backflow type irrigation needle tip is configured for sealer filling, controllability is increased, and a sealer is prevented from entering a human body. Accordingly, a cleaning degree and a therapy effect inside a root canal are greatly improved, and the reaction during and after an operation is reduced.

### BRIEF DESCRIPTION OF DRAWINGS

FIG. 1 is a flowchart of a manufacturing method for a backflow type irrigation needle tip according to a preferred example of the disclosure.

FIG. 2 is a schematic diagram of a manufacturing apparatus for a backflow type irrigation needle tip according to a preferred example of the disclosure.

FIG. 3 is a schematic diagram of adjusting an angle of a needle tip according to a preferred example of the disclosure.

FIG. 4 is a schematic sectional view of a backflow type irrigation needle tip acquired through a manufacturing method for according to a preferred example of the disclosure.

### DETAILED DESCRIPTION OF THE INVENTION

Embodiments of the disclosure are described below in combination with the drawings of the description. It should be noted that the embodiments involved in the description are not exhaustive, and do not represent the unique embodiments of the disclosure. The following corresponding examples are only for clearly describing the summary of the invention of the patent of invention, and are not intended to limit the embodiments of the disclosure. Those of ordinary skill in the art can also make changes and modifications in different forms on the basis of the description of the examples. Obvious changes or modifications that belong to the technical concept and the summary of the invention of the disclosure also fall within the scope of protection of the disclosure. Throughout the description, a portion “comprising” a constituent element means that it can also include other constituent elements, and does not exclude other constituent elements, unless specifically stated on the contrary.

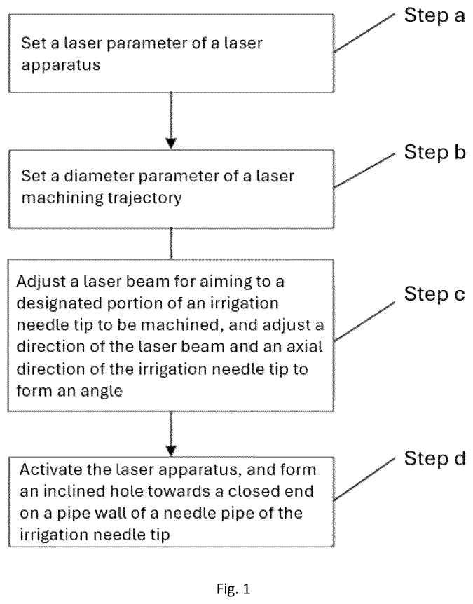

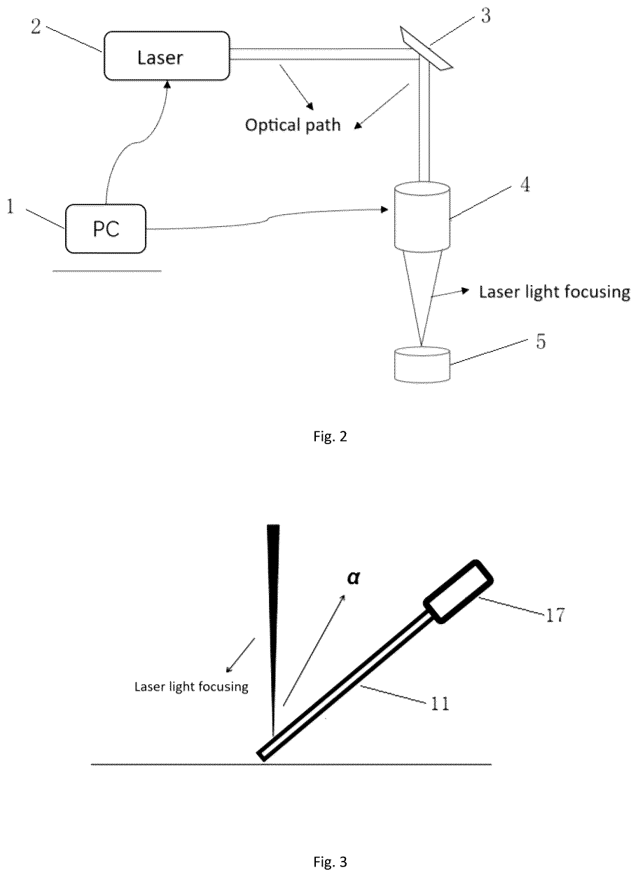

As shown in FIG. 1, a flowchart of a manufacturing method for a backflow type irrigation needle tip according to the disclosure is shown. As shown in FIG. 2, a schematic diagram of a manufacturing apparatus for a backflow type irrigation needle tip according to a preferred example of the disclosure is shown. As shown in FIG. 3, a schematic diagram of adjusting an angle of a needle tip according to a preferred example of the disclosure is shown. The manufacturing method for a backflow type irrigation needle tip according to a preferred example of the disclosure is described in detail below in conjunction with FIGS. 1-3.

In the method of the disclosure, a hole is punched on the irrigation needle tip through a laser apparatus, and a pointed portion of the irrigation needle tip is a closed end. The method includes steps a-d:

Step a: a laser parameter of the laser apparatus is set, where the laser parameter includes a repetition frequency, single pulse energy, and a focal spot size. The laser apparatus herein includes a laser 2, an optical path reflection mirror 3, and a machining head 4. The laser 2 is configured to emit out a laser beam. The optical path reflection mirror 3 is configured to reflect the laser beam, so as to adjust an optical path. The machining head 4 is configured to focus a laser light on the irrigation needle tip to be machined. The irrigation needle tip to be machined (not shown in FIG. 1) is fixed to a workbench 5. The laser 2 and the machining head 4 are controlled through a computer 1. The laser parameter is set according to requirements in a process file. For example, the laser light emitted by the laser has a wavelength of 100 nm-1200 nm, a pulse width of 100 ns-10 fs, a pulse frequency of 1 KHz-2000 KHz, and single pulse energy of a uj magnitude, which may be specifically adjusted according to a metal material of the irrigation needle tip, a thickness of a pipe wall, an inclination angle of the hole, and a hole diameter.

Step b: a diameter parameter of a laser machining trajectory is set. The parameter is set according to requirements of a technical parameter. Preferably, a suitable machining head 4 is selected, so that a focal spot generated after the laser light passes through the machining head 4 is 10 microns-30 microns, a specific value of which is related to the size of the hole. The machining trajectory of the machining head 4 is precisely controlled through the computer 1. In a preferred example herein, the laser machining trajectory is circular, and has a diameter of 90 microns-400 microns. Preferably, the diameter is 90 microns. In other words, the inclined hole formed on the irrigation needle tip is a circular hole having a diameter of 90 microns. In other examples, the laser machining trajectory may be elliptical, and the inclined hole formed on the irrigation needle tip is an elliptical hole accordingly.

Step c: a laser beam for aiming is adjusted to a designated portion of the irrigation needle tip to be machined, and a direction of the laser beam and an axial direction of the irrigation needle tip are adjusted to form an angle, where a range of the angle α is 10°≤α<90°.

A distance from the designated portion to the pointed portion of the irrigation needle tip in the step is 0.07 millimeter-16 millimeters. The designated portion is a position where the inclined hole is formed on the needle pipe of the irrigation needle tip. Specifically, 0.07 millimeter is a minimum wall thickness of an existing irrigation needle tip. Moreover, 16 millimeters is a length of a threaded portion of a root canal file, and corresponds to a maximum distance from a lateral hole to the pointed portion of the irrigation needle tip. Generally, the closer the inclined hole is to the pointed portion, the better the effect of solution backflow and irrigation is. Therefore, preferably, the distance from the designated portion to the pointed portion of the irrigation needle tip may be 0.07 millimeter-10 millimeters. More preferably, the distance from the designated portion to the pointed portion of the irrigation needle tip may be 0.07 millimeter-5 millimeters.

In addition, it is required to form a certain angle between the direction of the laser beam and the axial direction of the irrigation needle tip (an axial direction of the needle pipe). Specifically, the irrigation needle tip is fixed to an auxiliary workpiece of the workbench 5, and a desired angle α is acquired by adjusting an angle between a surface of the auxiliary workpiece and a horizontal plane. Preferably, a range of the included angle α between the direction of the laser beam and the axial direction of the needle tip is 40°≤α<70°. In a typical example, the included angle between the direction of the laser beam and the axial direction of the needle tip may be 45°. As shown in FIG. 3, in which the direction of the laser beam is vertically downward, the included angle of 45° is formed between the axial direction of the irrigation needle tip 11 and the laser beam. In other examples, for example, in a case that the root canal is internally concaved or internally absorbed, and a pulp horn is not fully opened, the range of the included angle α between a hole forming direction of the inclined hole and the axial direction of the needle tip is 10°≤α≤20°. Accordingly, a backflow irrigant at such an angle has a desirable irrigation effect.

Finally, step d: the laser apparatus is activated, and the inclined hole towards the closed end is formed on a pipe wall (the side wall) of the needle pipe of the irrigation needle tip. An included angle between an orientation (the hole forming direction) of the inclined hole and the axial direction of the needle tip equals the foregoing angle α.

Preferably, 1-6 inclined holes may be provided. In a preferred example, 1 inclined hole may be provided. In another preferred example, 2 or 4 inclined holes 14 are provided, and the 2 or 4 inclined holes may be positioned on two sides of a pipe wall 3 of the irrigation needle tip. In yet another preferred example, 3 or 6 inclined holes 14 are provided, and the 3 or 6 inclined holes may be positioned on a pipe wall (a side wall) of the irrigation needle tip at 120 degrees from one another in a radial circumference direction.

As described above, the disclosure provides the manufacturing method for a backflow type irrigation needle tip innovatively. In the method of the disclosure, the hole is punched on the side wall of the needle pipe of the irrigation needle tip by the laser beam controlled through a computer system, so that a position, size, and direction of the inclined hole can be precisely designed and controlled. In other words, in the method of the disclosure, water outlet holes having different numbers, sizes, and directions may be punched on the side wall of the needle pipe of the irrigation needle tip according to clinical demands. Water outlet holes may also be individually manufactured according to different root canal shapes. In this way, infected and necrotic tissue of each portion of the root canal and residual pulp debris can be irrigated, drained, and discharged through a crown, instead of being rushed out of an apical foramen.

According to the disclosure, the hole is punched on the material of the irrigation needle tip through the laser light. The small hole punched has a desirable quality. Especially when a plurality of small identical holes are punched, their uniform size and shape can be ensured. Accordingly, a hole punching speed is high, and production efficiency is high. Moreover, a diameter of these small holes can be as small as dozens of microns, which can fully satisfy machining requirements.

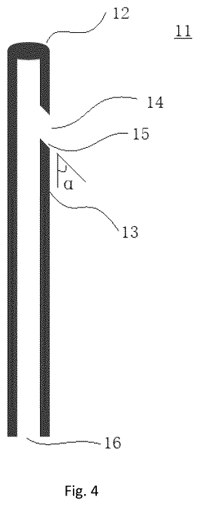

As shown in FIG. 4, a backflow type irrigation needle tip 11 manufactured through the method of the disclosure is shown, where a pointed portion 12 of the irrigation needle tip 11 is a closed end. The other end 16 opposite the closed end of the irrigation needle tip 11 is detachably connected to a syringe 17 (shown in FIG. 3), typically through a needle seat. An inclined hole 14 towards the closed end 12 is formed on a pipe wall (an outer wall) 13 of the irrigation needle tip 11, and an included angle between an orientation (a hole forming direction) 15 of the inclined hole 14 and an axial direction of the needle tip equals the foregoing angle α, and a range of the angle α is: 10°≤α≤90°. Preferably, the range of the angle α between the hole forming direction 15 of the inclined hole and the axial direction of the needle tip is 40°≤α≤70°. More preferably, the included angle between the hole forming direction 15 of the inclined hole 14 and the axial direction of the needle tip may be 45°. In other examples, for example, in a case that a root canal is internally concaved or internally absorbed, and a pulp horn is not fully opened, the range of the included angle α between the hole forming direction 15 of the inclined hole and the axial direction of the needle tip is 10°≤α≥200. Accordingly, a backflow irrigant at such an angle has a desirable irrigation effect.

Preferably, a distance from the inclined hole 14 and the pointed portion 12 of the irrigation needle tip is 0.07 millimeter-16 millimeters. Generally, the closer the inclined hole 14 is to the pointed portion 12, the better the effect of solution backflow is. Preferably, the distance from the inclined hole 14 to the pointed portion 12 of the irrigation needle tip may be 0.07 millimeter-10 millimeters. More preferably, the distance from the inclined hole 14 to the pointed portion 12 of the irrigation needle tip may be 0.07 millimeter-5 millimeters.

Preferably, the inclined hole 14 is a circular hole. A hole diameter of the inclined hole 14 is 90 microns-200 microns. In a preferred example, the hole diameter of the inclined hole 14 is 90 microns. In other examples, the inclined hole 14 may be an elliptical hole, etc.

1-6 inclined holes 14 may be provided. In the example shown in FIG. 4, 1 inclined hole 14 is provided. In another preferred example, 2 inclined holes 14 are provided, and the 2 inclined holes may be positioned on two sides of the pipe wall 13 of the irrigation needle tip. In yet another preferred example, 3 inclined holes 14 are provided, and the 3 inclined holes may be positioned on the pipe wall 13 of the irrigation needle tip at 120 degrees from one another in a radial circumference direction.

As described above, in the backflow type irrigation needle tip manufactured through the method of the disclosure, the above inclined hole 14 is provided on the pipe wall 13 of the irrigation needle tip 11. After flowing into the closed end 12 along the pipe wall 13 of the irrigation needle tip 11, the solution flows back after encountering an obstruction, and is ejected out of the inclined hole 14. An ejection direction of the solution is controlled through the hole forming angle of the inclined hole 14, and the effect of solution backflow is realized through an inclined hole based flow guide effect.

By changing the hole forming angle of the inclined hole of the needle tip and a flow direction of the irrigant, the backflow type irrigation needle tip manufactured through the method of the disclosure may generate a large irrigation pressure and a high flow speed. Therefore, requirements of a large amount of rapid and safe irrigation can be satisfied, and a cleaning degree and a therapy effect inside the root canal can be greatly improved. Moreover, the backflow type irrigation needle tip manufactured through the method of the disclosure may form or increase a local negative pressure at a pointed portion position of a solution backflow and ejection point. Accordingly, an internal pressure of structures such as a root tip and a bone marrow cavity is affected, and an irrigant, an inflammatory exudate, etc. dissolved with the infected and necrotic tissue are led to be discharged through a crown of the root canal, instead of entering a body. When a sealer is injected into the root canal through the irrigation needle tip manufactured through the method of the disclosure, the material can also be prevented from entering the internal structures, such as the bone marrow cavity, a maxillary sinus, and a neural canal at a relatively negative pressure.

The disclosure can obviously improve two most significant effects in root canal irrigation when applied to root canal therapy. The first is a chemical effect, in other words, biofilms are chemically destructed or inactivated, microorganisms are killed, endotoxin is inactivated, and residual dental pulp tissue, a dentin smear layer, etc. are dissolved. The effect can be implemented only through a chemically active solution (such as sodium hypochlorite). It has been reported that a specific bacterial biofilm is required to be soaked in a sodium hypochlorite solution having a concentration of 6% for 15 minutes, so as to be dissolved. The sodium hypochlorite solution can play the biggest role only if it is continuously replenished and renewed in a narrow root canal space. The irrigation needle tip of the disclosure generates an effective reverse water flow, so as to improve renewal efficiency of the irrigant inside the root canal, especially at a tip of the root canal (renewal from a root tip to the crown) in a case that a minimum pressure at an apical foramen is ensured. The second is a mechanical effect, in other words, a force applied through the irrigant mechanically destroys, strips, and removes microorganisms/biofilms, residual dental pulp tissue, dentin debris, etc. in the root canal system. The mechanical effect can be applied through the irrigant (for example, a solution containing water and a disinfectant or chelated drug). In a process of irrigation, the reverse and high-speed flowing solution generates an impact force and a friction force on a root canal wall. Therefore, the bacterial biofilms, residual dental pulp tissue, dentin debris, etc. are separated from the root canal wall and discharged through the crown, and the periapical inflammatory exudate is drained. Finally, the root canal is filled with a clear and disinfectant irrigant. Thus, root canal drying and filling can be performed because infection control is a crucial factor in root canal therapy. Without any additional apparatus or special training for dentists, the disclosure ensures the effect of single root canal therapy, and also conforms to the concept of comfortable, efficient, and safe therapy.

In addition, a use range of the backflow type irrigation needle tip manufactured through the method of the disclosure is not limited to the inside of the root canal. The backflow type irrigation needle tip is also applicable to various fine tissue structures, such as pericoronal and periodontal pockets. The infected and necrotic tissue can be discharged from the body through reverse irrigation, so as to promote wound healing. No pressures of the irrigant and a filling material are released to a deep portion of the tissue. Accordingly, adverse consequences such as acute pains and tissue damage are avoided, tension of a patient is relieved to a certain extent, and a doctor-patient relation is more harmonious.

Obviously, those of ordinary skill in the art should recognize that the above examples are merely used to illustrate the disclosure, and are not intended to limit the disclosure. Changes and variations to the above examples will fall within the scope of the claims of the disclosure without departing from the scope of essential spirits of the disclosure.

What is claimed is:

1. A manufacturing method for a backflow type irrigation needle tip, wherein a hole is punched on the irrigation needle tip through a laser apparatus, and a pointed portion of the irrigation needle tip is a closed end; and the method comprises:
step a: setting a laser parameter of the laser apparatus, wherein the laser parameter comprises a repetition frequency, single pulse energy, and a focal spot size;
step b: setting a diameter parameter of a laser machining trajectory;
step c: adjusting a laser beam for aiming to a designated portion of the irrigation needle tip to be machined, and adjusting a direction of the laser beam and an axial direction of the irrigation needle tip to form an angle, wherein a range of the angle α is 10°≤α<90°; and
step d: activating the laser apparatus, and forming an inclined hole towards the closed end on a pipe wall of the irrigation needle tip.

2. The manufacturing method for a backflow type irrigation needle tip according to claim 1, wherein in step a, the laser apparatus comprises a laser, an optical path reflection mirror, and a machining head.

3. The manufacturing method for a backflow type irrigation needle tip according to claim 2, wherein the laser and the machining head are controlled through a computer.

4. The manufacturing method for a backflow type irrigation needle tip according to claim 3, wherein in step b, the laser machining trajectory is circular and has a diameter of 90 microns-400 microns.

5. The manufacturing method for a backflow type irrigation needle tip according to claim 4, wherein in step c, a distance from the designated portion to the pointed portion of the irrigation needle tip is 0.07 millimeter-16 millimeters.

6. The manufacturing method for a backflow type irrigation needle tip according to claim 5, wherein the range of the angle α is 40°≤α≤70°.

7. The manufacturing method for a backflow type irrigation needle tip according to claim 1, wherein in step c, the irrigation needle tip is fixed to an auxiliary workpiece of a workbench, and a desired angle α is acquired by adjusting an angle between a surface of the auxiliary workpiece and a horizontal plane.

8. A backflow type irrigation needle tip, manufactured through the manufacturing method according to claim 1, wherein a pointed portion of the irrigation needle tip is a closed end, an inclined hole towards the closed end is formed on a pipe wall of the irrigation needle tip, and a range of an included angle α between a hole forming direction of the inclined hole, i.e. a direction of a laser beam and an axial direction of the needle tip is 10°≤α≤90°.

9. The backflow type irrigation needle tip according to claim 8, wherein the included angle α between the hole forming direction of the inclined hole and the axial direction of the needle tip is 45°.

10. The backflow type irrigation needle tip according to claim 8, wherein a distance from the inclined hole to the pointed portion of the irrigation needle tip is 0.07 millimeter-16 millimeters, and 1-6 inclined holes are provided.
